(19) United States
(12) Patent Application Publication
(10) Pub. No.: US 2011/0003897 A1
(43) Pub. Date: Jan. 6, 2011

(54) METHODS OF ENGINEERING POLAR DRUG PARTICLES WITH SURFACE-TRAPPED HYDROFLUOROALKANE-PHILES

(75) Inventors: Sandro R.P. Da Rocha, Troy, MI (US); Libo Wu, Detroit, MI (US)

Correspondence Address: C. Rachal Winger;Intellectual Property Docketing Department, 925 Fourth Avenue, Suite 2900, Seattle, WA, 98104-1158, US

(73) Assignee: WAYNE STATE UNIVERSITY, Detroit, MI (US)

(21) Appl. No.: 12/741,021

(22) PCT Filed: Oct. 31, 2008
(86) PCT No.: PCT/US2008/082105

§ 371 Date: Sep. 14, 2010

Related U.S. Application Data
(60) Provisional application No. 60/984,973, filed on Nov. 2, 2007.

(51) Int. Cl. A61K 31/138; A61P 11/06

(52) U.S. Cl. 514/653

(57) ABSTRACT

Disclosed herein are polar drug particles with surface-trapped hydrofluoroalkane-philes and methods of making the same.

### CROSS-REFERENCES TO RELATED APPLICATIONS

This application is a 371 national phase of International Application No. PCT/US2008/082105 filed Oct. 31, 2008, which is a non-provisional of U.S. Provisional Application No. 60/984,973 filed on Nov. 2, 2007, the entire disclosures of which are incorporated herein by reference.

### STATEMENT REGARDING FEDERALLY SPONSORED RESEARCH

This work was partially funded by National Science Foundation grant NSF-CBET No. 0553537, and the United States government has, therefore, certain rights to the present invention.

### FIELD OF THE DISCLOSURE

Disclosed herein are polar drug particles with surface-trapped hydrofluoroalkane-philes and methods of making the same.

### INTRODUCTION

Pressurized metered dose inhalers (pMDIs) are the most widely used devices for pulmonary drug delivery (Courrier et al., 2002). While chlorofluorocarbons (CFCs) were employed as the propellants in pMDI formulation for decades (McDonald and Martin, 2000), concerns about their ozone depletion potential has prompted the search for more environmentally friendly alternatives (Noakes, 1995). The biocompatible, non-ozone depleting hydrofluoroalkanes (HFAs) have been selected as the replacements to CFCs (Vervaet and Byron, 1999). Whereas HFAs and CFCs have similar densities and vapor pressures, several of their physicochemical properties are significantly different (Blondino and Byron, 1998). As a consequence, many CFC-based formulations were found not to be compatible with HFA propellants.

There are two basic types of pMDI formulations: (i) solution-based, in which the active ingredients are dissolved in the propellant; and (ii) dispersion-based, where the active ingredients are suspended in the propellant. Dispersions are inherently unstable due to the cohesive forces between particles, and due to the gravitational fields (Rogueda, 2005). Therefore, surface active agents are generally required in order to provide stability to the drug suspension (Courrier et al., 2002; Rogueda, 2005). However, due to the different solvent properties between CFCs and HFAs, surfactants used in CFC-based, FDA-approved formulations have extremely low solubility in HFAs (Courrier et al., 2002). In order to overcome the surfactant solubility issues, co-solvents are generally employed (Vervaet and Byron, 1999). The use of co-solvents is not always possible as they may cause adverse effects such as a decrease in the overall chemical and physical stability of the formulation (Tzou et al., 1997).

Such difficulties have prompted the research community not only to design amphiphiles that have enhanced solubility in cosolvent-free HFAs (James, 2002; Rogueda, 2005; Traini et al., 2006; Wu and da Rocha, 2007), but also to develop novel formulations altogether (Dickinson et al., 2001; Edwards et al., 1997; Jones et al., 2006; Liao et al., 2005; Rogueda, 2005; Selvam et al., 2006; Steckel and Wehle, 2004; Wu et al., 2007a). Many of the advances related to novel dispersion formulations are centered on controlling the morphology or the surface properties of the drug particles in an attempt to minimize the forces that impart physical instability to the system (Dickinson et al., 2001; Edwards et al., 1997; Williams and Liu, 1999). For example hollow porous particles of cromolyn sodium and salbutamol sulfate obtained by spray-drying possess excellent physical stability in HFAs compared with the commercial formulations (micronized drug crystals) (Dellamary et al., 2000). However, the content of active drug ingredients accounts for only 50 wt % or less in the spray dried powder. Dispersion formulations of nanometer-sized salbutamol sulfate particles obtained by lyophilization of lecithin stabilized water-in-hexane emulsions have been also reported (Dickinson et al., 2001). One of the shortcomings of that approach is that the drug particles can be suspended in HFAs only in the presence of hexane as co-solvent.

Central to the development of novel approaches for dispersion-based pMDIs is the measurement of particle-particle interactions. Macroscopic information regarding colloidal stability of dispersion-based formulations can be assessed by sedimentation rate experiments (Ranucci et al., 1990). However, to precisely evaluate and compare the effectiveness of the proposed modifications of the drug particles surface characteristics/chemistry or of novel surface active ingredients, quantitative characterization of particle-particle interaction is needed. Colloidal Probe Microscopy (CPM), a variation of the Atomic Force Microscopy (AFM) (Butt et al., 2005), is especially suited for this purpose. Several groups have taken advantage of this powerful technique, and have investigated the effect of different additives on systems and drugs relevant to pMDI formulations (Ashayer et al., 2004; Traini et al., 2006; Wu and da Rocha, 2007; Young et al., 2003). The CPM results serve not only to directly probe the effect of the different excipients/surface property modifications, but also allow decoupling of the confounding information regarding the physical stability of the dispersions and the aerosol properties of the formulation that may also be affected by the device components and other formulation parameters (Tzou et al., 1997; Vervaet and Byron, 1999).

### SUMMARY OF THE DISCLOSURE

The present disclosure provides methods for creating stable dispersions of polar drugs in propellant HFAs. The approach consists of ‘trapping’ HFA-philic groups at the particle surface in a way that they can act as stabilizing agents, thus preventing flocculation of the otherwise unstable colloidal drug particles. This approach has advantages compared to surfactant-stabilized colloids in that no free stabilizers remain in solution (reduced toxicity), and the challenges associated with the synthesis of well-balanced amphiphiles are circumvented (Wu and da Rocha, 2007). A modification of the emulsification-diffusion technique (Leroux, 1995) for preparing the drug particles was used. The approach was tested by forming amorphous polyethylene (PEG)-“coated” salbutamol sulfate (SS) and terbutaline hemisulfate (THS) particles. CPM was employed to measure the forces between bare-and PEG-modified drug particles in 2H,3H-perfluoropentane (HPFP), a mimic to propellant HFAs (Ashayer et al., 2004; Rogueda, 2003; Traini et al., 2006; Wu et al., 2007b), thus quantitatively assessing the effect of the surface modification. The effect of formulation parameters on the morphology of the drug particles prepared by emulsification-diffusion was also evaluated. The physical (bulk) stability of the dispersions in 1,1,1,2-tetrafluoroethane (HFA134a) and 1,1,1,2,3,3,3heptafluoropropane (HFA227), and the performance of the corresponding aerosols were also studied.

Embodiments disclosed herein include a method of producing a stable dispersion of a polar drug in hydrofluoroalkane (HFA) by providing a polar drug particle; and adding a quantity of the HFA to the polar drug particle to produce the stable dispersion. In another embodiment, the polar drug is a pulmonary drug. In yet another embodiment, the pulmonary drug is salbutamol sulfate or terbutaline hemisulfate. In yet another embodiment, the pulmonary drug is a drug for the treatment of asthma. In yet another embodiment, the HFA is selected from the group consisting of 1,1,1,2-tetrafluoroethane, 1,1,1,2,3,3,3-heptafluoropropane, and combinations thereof.

In yet another embodiment the method further includes sonicating the HFA and polar drug particle. In yet another embodiment the polar drug particle is produced by emulsification-diffusion. In another embodiment, the polar drug particle is selected from the group consisting of a polar drug particle without a stabilizing agent, a particle-stabilized polar drug particle, an HFA-philic moiety-modified, particle-stabilized polar drug particle and combinations thereof.

In yet another embodiment, the polar drug particle without the stabilizing agent is produced by dissolving the polar drug in water to form an aqueous solution, adding the aqueous solution to a first quantity of ethyl acetate, then emulsifying the aqueous solution and ethyl acetate to form a water-in-ethyl acetate (W/Ac) emulsion, and then transferring the W/Ac emulsion to a second quantity of ethyl acetate whereby the polar drug particle without said stabilizing agent is formed.

In another embodiment, the particle-stabilized polar drug particle is produced by providing an aqueous dispersion of a stabilizing particle, dissolving the polar drug in the aqueous dispersion to form a polar drug and stabilizing particle dispersion, then adding the polar drug and stabilizing particle dispersion to a first quantity of ethyl acetate and emulsifying to form a water-in-ethyl acetate (W/Ac) emulsion, and then transferring the W/Ac emulsion to a second quantity of ethyl acetate, whereby said particle-stabilized polar drug particle is formed. In yet another embodiment, the particle is lecithin.

In another embodiment, the HFA-philic moiety-modified, particle-stabilized polar drug particle is produced by providing an aqueous dispersion of a stabilizing particle, dissolving a quantity of the HFA-philic moiety and the polar drug in the aqueous dispersion of the stabilizing particle to form a HFA-philic moiety, polar drug and stabilizing particle dispersion, then adding the HFA-philic moiety, polar drug and stabilizing particle dispersion to a first quantity of ethyl acetate, emulsifying to form a water-in-ethyl acetate (W/Ac) emulsion, and then transferring the W/Ac emulsion to a second quantity of ethyl acetate, whereby said HFA-philic moiety-modified, particle-stabilized polar drug particle is formed. In yet another the HFA-philic moiety is a polyethylene (PEG).

Embodiments disclosed herein also include a composition having a stable dispersion of a polar drug in hydrofluoroalkane (HFA). In another embodiment, the polar drug is a pulmonary drug. In yet another embodiment the pulmonary drug is salbutamol sulfate or terbutaline hemisulfate. In yet another embodiment, the pulmonary drug is a drug for the treatment of asthma. In yet another embodiment, the HFA is selected from the group consisting of 1,1,1,2-tetrafluoroethane, 1,1,1,2,3,3,3-heptafluoropropane, and combinations thereof.

Embodiments disclosed herein also include polar drug particles, including polar drug particles without a stabilizing agent, particle-stabilized polar drug particles, and hydrofluoroalkane(HFA)-philic moiety-modified, particle-stabilized polar drug particles. The polar drug particles without a stabilizing agent are produced by dissolving the polar drug in water to form an aqueous solution, adding the solution to a first quantity of ethyl acetate and emulsifying to form a water-in-ethyl acetate (W/Ac) emulsion, then transferring the W/Ac emulsion to a second quantity of ethyl acetate, whereby said polar drug particle without the stabilizing agent is formed.

The particle-stabilized polar drug particles are produced by providing an aqueous dispersion of a stabilizing particle, dissolving the polar drug in the aqueous dispersion to form a polar drug and stabilizing particle dispersion, then adding the dispersion to a first quantity of ethyl acetate and emulsifying to form a water-in-ethyl acetate (W/Ac) emulsion, and then transferring the W/Ac emulsion to a second quantity of ethyl acetate, whereby the particle-stabilized polar drug particle is formed.

The hydrofluoroalkane(HFA)-philic moiety-modified, particle-stabilized polar drug particles are produced by providing an aqueous dispersion of a stabilizing particle, dissolving a quantity of the HFA-philic moiety and the polar drug in the aqueous dispersion of the stabilizing particle to form a HFA-philic moiety, polar drug and stabilizing particle dispersion, then adding the dispersion to a first quantity of ethyl acetate and emulsifying to form a water-in-ethyl acetate (W/Ac) emulsion, and then transferring the W/Ac emulsion to a second quantity of ethyl acetate, whereby the HFA-philic moiety-modified, particle-stabilized polar drug particle is formed.

### BRIEF DESCRIPTION OF THE FIGURES

FIG. 1 shows SEM of the (a) commercial SS crystals as received; and the SS spheres prepared by emulsification-diffusion technique at (b) 303 K and 0.8:19 water to ethyl acetate volume ratio (W:Ac, ml), (c) 316 K and 0.8:19 W:Ac; (d) 311 K and 0.8:14 W:Ac; (e) 311 K and 0.8:19 W:Ac; and (f) 311 K and 0.8:24 W:Ac.

FIG. 2 shows XRD spectrum of commercial SS crystals, and SS spheres prepared using the emulsification-dilution technique.

FIG. 3 shows images of the water-in-ethyl acetate emulsions (40:60 W:Ac in volume) 5 min after mechanical energy was stopped: (a) no stabilizing agent; and (b) lecithin-stabilized emulsion −5 mg·ml-I dispersion.

FIG. 4 shows the effect of lecithin concentration on the interfacial tension of the waterethyl acetate interface at 298 K.

FIG. 5 shows SEM micrographs of (a) SS spheres prepared from lecithin-stabilized water-in-ethyl acetate emulsions at 311 K and 0.8:19 W:Ac volume ratio; (b) PEG300-modified SS spheres obtained from lecithin-stabilized water-in-ethyl acetate emulsions at same temperature and volume ratio as in (a). Inset: PEG-SS before hexane washing

FIG. 6 shows IH NMR spectra of (a) commercial SS; (b) PEG300-modified SS spheres prepared from lecithin-stabilized W/Ac emulsions. Peak at 3.6 ppm is attributed to PEG.

FIG. 7 shows (a) SEM micrographs of PEG300-modified salbutamol sulfate (SS) sphere attached to an AFM probe. Inset: overhead view. (b) Adhesion force (Fad) histogram between bare SS (red distribution to the right of the diagram) and PEG-coated SS spheres (black distribution to the left of the diagram) in HPFP. Inset: average force curves for bare-SS and PEG300-modified SS particles. The green lines represent the Gaussian fit of the histograms.

FIG. 8 shows Dispersion stability of SS spheres in HFA134a at 298 K and saturation pressure. (a) SS spheres from emulsification-diffusion technique (average diameter of 550 nm); (b) SS spheres from lecithin-stabilized emulsions (average diameter 350 nm); (c) PEG300 modified SS spheres from lecithin-stabilized emulsions (average diameter 450 nm). Results for the suspension stability of SS particles in HFA 134a and HFA227 are very similar.

FIG. 9 shows Aerodynamic particle size distribution of [VENTOLIN® HFA (GlaxoSmithKline (UK))], bare SS (diameter 550 nm), and PEG300-modified SS (diameter 450 nm) formulations in HFA134a (2 mg·ml-l) (a) without spacer; (b) with spacer. (AC, IP, SP and F refer to actuator plus valve stem, induction port, spacer and terminal filter respectively).

### DETAILED DESCRIPTION

The present disclosure provides methods for creating stable dispersions of polar drugs in propellant HFAs. The approach consists of ‘trapping’ HFA-philic groups at the particle surface in a way that they can act as stabilizing agents, thus preventing flocculation of the otherwise unstable colloidal drug particles. This approach has advantages compared to surfactant-stabilized colloids in that no free stabilizers remain in solution (reduced toxicity), and the challenges associated with the synthesis of well-balanced amphiphiles are circumvented (Wu and da Rocha, 2007). A modification of the emulsification-diffusion technique (Leroux, 1995) for preparing the drug particles was used. The approach was tested by forming amorphous polyethylene (PEG)-“coated” salbutamol sulfate (SS) and terbutaline hemisulfate (THS) particles. CPM was employed to measure the forces between bare-and PEG-modified drug particles in 2H,3H-perfluoropentane (HPFP), a mimic to propellant HFAs (Ashayer et al., 2004; Rogueda, 2003; Traini et al., 2006; Wu et al., 2007b), thus quantitatively assessing the effect of the surface modification. The effect of formulation parameters on the morphology of the drug particles prepared by emulsification-diffusion was also evaluated. The physical (bulk) stability of the dispersions in 1,1,1,2-tetrafluoroethane (HFA134a) and 1,1,1,2,3,3,3heptafluoropropane (HFA227), and the performance of the corresponding aerosols were also studied.

### Examples

2. Materials and Methods

2.1. Materials

Polyethylene glycol (PEG) (300 MW) was purchased from Aldrich Chemicals Ltd. 2H,3Hperfluoropentane (HPFP, 98%) was purchased from SynQuest Labs Inc. Pharma grade hydrofluoroalkanes (HFA134a and HFA227, assay>99.99%) were kindly donated by Solvay Fluor and Derivate GmbH & Co. (Hannover-Germany). Salbutamol sulfate (SS) was purchased from Spectrum Chemicals. Terbutaline hemisulfate (THS) salt was purchased from Sigma. Lecithin (refined) was from Alfa Aesar. All the other organic solvents used in this work were supplied by Fisher Chemicals and were of analytical grade. Deionized water (NANOpure® Dlamond™ UV ultrapure water system: Barnstead International, Dubuque, Iowa), with a resistivity of 18.2 and surface tension of 73.8 mN·m-I at 296 K, was used in all experiments. Two-component Epoxy (Epotek 387) was purchased from EPO-TEK. Si3N4 contact mode cantilevers with integrated pyramidal tips (NP-20) were purchased from Veeco Instruments.

2.2. Preparation of Polar Drug Particles by Emulsification-Diffusion

Emulsions without Stabilizing Agents. Polar drug particles were prepared by emulsification-diffusion. Briefly, 25 mg of the drug was dissolved in 0.8 ml of water. This aqueous solution was then added to a known amount of ethyl acetate. After equilibration the system was emulsified using a sonication bath (VWR, P250D). Mechanical energy was input to the system for 15 min, with the power level set to 180 W. Immediately after sonication was stopped, the water-in-ethyl acetate (W/Ac) emulsion was transferred into a large volume (150 ml) of ethyl acetate. Because of the high solubility of water in ethyl acetate (Hefter, 1992), water diffuses out of the dispersed phase (emulsion droplets), and into bulk ethyl acetate, forming the drug particles as templated by the droplets. Particles were prepared at temperatures above room temperature to facilitate the control of the system temperature during the particle formation process. The drug particles were collected by centrifugation, and subsequently dried at room temperature.

Particle-Stabilized Emulsions. Commercial lecithin was first washed repeatedly with acetone and ethanol to obtain the treated lecithin powder, which was then dispersed in water at a concentration of 20 mg·ml-1, with the aid of a sonication bath. The size and distribution of the lecithin particles dispersed in water were characterized by dynamic light scattering (DLS) (Brookhaven 90Plus particle size analyzer). W/Ac emulsions were formed by initially dissolving 25 mg of the polar drug in the 0.8 ml aqueous dispersion of lecithin. The dispersion was then emulsified in a known amount of ethyl acetate using a sonication bath for 15 min, and a power level of 180 W. The W/Ac emulsion was then transferred into 150 ml of ethyl acetate. Drug particles are formed by the mechanism discussed above. The particles were collected by centrifugation, washed with hexane twice to remove any residual lecithin, and then dried at room temperature.

PEG-modified, Particle-Stabilized Emulsions. To prepare the PEG-modified drug particles, a procedure similar to the one described above was employed. The only difference was that 200 mg of PEG300 was dissolved together with the 25 mg of drug in the aqueous dispersion of lecithin before formation of the W/Ac emulsion. Because PEG300 is soluble in both water and ethyl acetate, high initial concentration of PEG300 is required to guarantee that there would be enough PEG molecules trapped at the particles surface.

2.3. Characterization of the Drug Particles Formed by Emulsification-Diffusion

The shape, size and size distribution of the drug particles formed by the procedures described above were analyzed by scanning electron microscopy (SEM, Hitachi S-2400, Japan). After centrifugation, the particles were first dispersed in HPFP by sonication—for dilution of the sample. Drops of the drug dispersion in HPFP were placed onto cover glass slips and allowed to dry. The cover glass substrates were subsequently sputtered with gold for 30 s for SEM analysis. The particle size was obtained by direct observation of SEM images. On average, over 300 particles were measured for each micrograph. The morphology of the as received drug crystals, and those formed by emulsification-diffusion were determined with an X-ray Powder Diffractometer (Rigaku) with CuKa radiation (1.54 A.). The measured scatter angle (28) ranged from 5 to 80°. The composition and chemical stability of the particles were determined by IH NMR.

2.4. Preparation and Characterization of the AFM Probe Modified with Drug Particles

Single particles were glued onto silicon nitride contact-mode cantilevers (NP-20) with the help of AFM (Pico LE, Molecular Imaging). In brief, the two components of the epoxy (Epotek 377) were mixed and heated to 353 K in a water bath for 30 min, until it became highly viscous. A small drop of epoxy was then transferred onto a piece of silicon wafer. The AFM cantilever was first positioned above the drop of epoxy with the help of a CCD camera. The tip was then slowly brought into contact with the substrate until a very small amount of epoxy was transferred to the AFM tip. A similar procedure was used to attach a single drug particle to the tip of the AFM cantilever containing the epoxy. The drug-modified AFM tip was then kept at room temperature inside a desiccator for 24 h to allow complete curing of the epoxy. The spring constant of the drug-modified cantilever was determined using a module attached to AFM and the MI Thermal K 1.02 software (Wu et al., 2007b). SEM images of the modified cantilevers were obtained after the adhesion force measurements were performed.

2.5. Colloidal Probe Microscopy (CPM)

The cohesive force between drug particles was probed directly by CPM. CPM is an AFM-based technique where the force of interaction between a particle-modified AFM tip and another particle/substrate is measured in air/liquid, with 10-12 newton accuracy (Butt et al., 2005). Adhesion force (Fad) is defined as the product of the spring constant of the particle-modified AFM cantilever and the maximum cantilever deflection during the retraction stage of the force measurement. A fluid cell was used to conduct the CPM experiments in liquid HPFP at 298 K. Drug particles were initially deposited onto a silicon wafer from HPFP. The adhesive force between particle and the substrate is stronger than that between particles, so that the particles remain bound to the substrate during the measurements. Several particles randomly distributed on the substrate were selected for the Fad measurements. For each contact point between the two particles, 25 force-distance curves were recorded in a range of 2000 nm, and the sweep duration of 2 s. The histogram of the measured adhesion force (Fad) was fit to a Gaussian distribution, from which an average force and deviation were obtained (Wu and da Rocha, 2007; Wu et al., 2007b).

2.6. Interfacial Tension

The interfacial tension (y) between water (saturated with ethyl acetate) and ethyl acetate (saturated with water) in the presence of lecithin was measured using a pendant drop tensiometer described in for example, (Selvam et al., 2006) which is incorporated by reference herein for its teaching regarding the same. Measurements were carried out inside a sealed cuvette at 298 K. Because no experimental density values of the mutually saturated phases are available in the literature, the density of pure water and ethyl acetate was used to calculate the y.

2.7. Dispersion Stability in Propellant HFAs

An exact mass of the drug particles were initially fed into pressure proof glass vials (Catalog#: 68000318, West Pharmarceutical Services), and crimp-sealed with 50 metering valves (EPDM Spraymiser™, 3M Inc). Subsequently, a known amount of HFA (HFA134a or HFA227) was added with the help of a manual syringe pump (HiP 50-6-15) and a home-built high pressure aerosol filler, to a 2 mg·ml-l drug concentration in the propellant HFAs. The formulations were then sonicated in a low energy sonication bath (VWR, P250D, set to 180 W) for 10 min. The physical stability of the suspensions in HFAs was investigated by visually monitoring the dispersion as a function of the time elapsed after mechanical energy input was stopped.

2.8. Aerosol Characteristics

The aerosol properties of the pMDI formulations were determined with an Andersen Cascade Impactor (ACI, CroPharm, Inc.) operated at a flow rate of 28.3 L·min-l. The experiments were carried out at 298 K and 45% relative humidity. Before each test, several shots were first fired to waste, then 10 shots were released into the impactor, with an interval of 30 s between actuations. Three independent canisters were tested for each formulation. The average and standard deviation from those three independent runs are reported here. The drug deposited on the valve stem, actuator, induction port and stages were collected by thoroughly rinsing the parts with a known volume of 0.1 N NaOH aqueous solution. NaOH reacts with the model polar drug (salbutamol sulfate) to produce phenolate. This procedure is used to enhance the detection of salbutamol sulfate, which absorbs at the low end of the spectrum (225 nm) when in the sulfate form (Dellamary et al., 2000). The drug content was then quantified by UV spectroscopy, with a detection wavelength of 243 nm. The fine particle fraction (FPF) is defined as the percentage of drug on the respirable stages of the impactor (stage 3 to terminal filter) over the amount of drug released from the induction port to filter. The mass mean aerodynamic diameter (MMAD) is determined by plotting the results from the ACI (aerosol particle size vs. cumulative percentage less than the size range), on a log-probability scale, and interpolating for the value at 50 wt % of the aerosol size distribution. The geometric standard deviation (GSD) is defined as the square root of the ratio of 84.13% over 15.87% particle size distribution from the same graph described above, and indicates the particle size polydispersity (Smyth et al., 2004; Telko and Hickey, 2005; Williams et al., 2001). The effect of a spacer (Aerochamber Plus) on the aerosol characteristics was investigated. The results obtained with the formulations proposed here are contrasted with those obtained with VENTOLIN® HFA. The same actuator as that of VENTOLIN® HFA was used in all experiments.

3. Results and Discussion

3.1. Formation and Characterization of Drug Particles by Emulsification-Diffusion

Emulsification-diffusion has been extensively used in the preparation of organic particles, usually polymers (Choi et al., 2002; Galindo-Rodriguez, 2004; Kwon, 2001; Leroux, 1995; Quintanar-Guerrero et al., 1996; Trotta et al., 2004). Because of the hydrophobic nature of those solutes, the morphology of the emulsions was typically oil-in-water (Choi et al., 2002; Kwon, 2001; Leroux, 1995; Quintanar-Guerrero et al., 1996). Considerably less attention has been given to the formation of particles of water soluble compounds using this approach (Trotta et al., 2004). In this work the applicability of the emulsification-diffusion technique for generating particles of polar drugs that are relevant to HFA-based pMDI formulations was demonstrated. How the morphology (size, size distribution) and surface characteristics of polar drug particles can be tuned in order to generate stable dispersions in HFA propellants was shown. Salbutamol sulfate (SS) was chosen as a model polar drug due to its medical relevance (Boskabady and Saadatinejad, 2003). To demonstrate the applicability of the methodology proposed here to other polar drugs, results for terbutaline hemisulfate (THS) are also reported. THS is often used in short-term treatment of asthma (Lafrate et al., 1986).

3.1.1. Particles from Emulsions without Stabilizing Agents. In order to prepare polar drug particles by emulsification-diffusion, an aqueous solution of drug is first emulsified in ethyl acetate by sonication, thus forming a water-in-ethyl acetate (W/Ac) emulsion. Because of the low interfacial tension (y) between water and ethyl acetate (6.8 mN·m-l at room temperature) (Donahue and Bartell, 1952), an emulsion is easily formed even in the absence of surface active agents. The W/Ac emulsion was subsequently added into a large volume of ethyl acetate. The water in the dispersed phase diffuses into the bulk ethyl acetate due to the high solubility of water in that solvent (Hefter, 1992). As water leaves the emulsion droplets, a supersaturation condition of the drug is reached. Particles nucleate and grow within the emulsion droplets, which serve as templates. The growing nuclei are arrested within the droplets, thus giving origin to spherical particles.

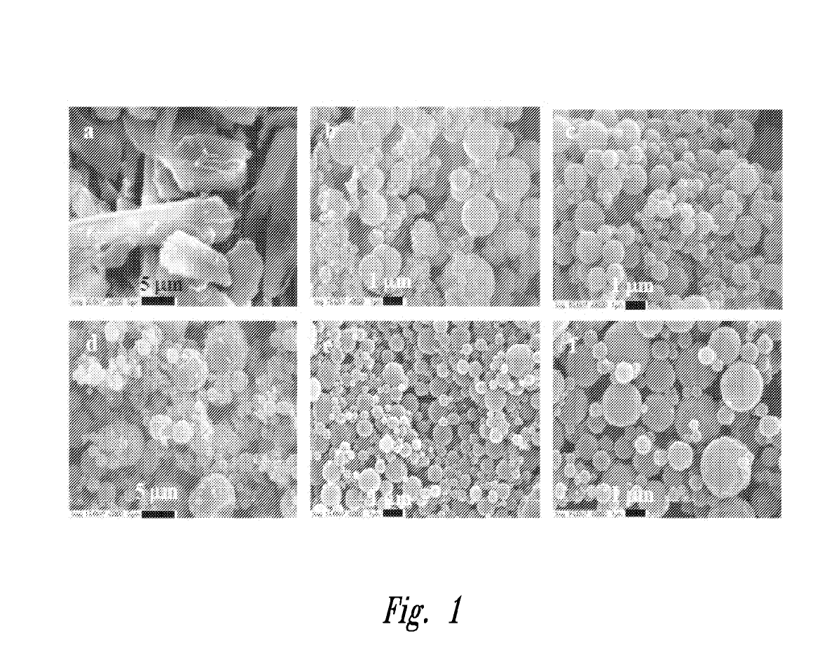

In this work the effect of various parameters on the size and morphology of SS spheres prepared by emulsification-diffusion, including the initial water to ethyl acetate (W:Ac) volume ratio (ratio before exposing the emulsion to a large excess of ethyl acetate), and the temperature was investigated. The ability to control the surface chemistry, size and size distribution of drug particles used in pMDIs and other inhalation formulations is of great relevance because they affect the cohesive interaction between drug particles, and the physical stability (Rogueda, 2005). FIG. 1 shows the SEM images of SS spheres prepared at different temperatures and various W:Ac ratios. The images reveal that the SS particles are nearly spherical, smooth, and are polydisperse. The average diameter of the spheres as a function of the emulsification temperature and W:Ac volume ratio estimated from the SEM micrographs is summarized in Table 1.

TABLE 1
Effect of emulsification temperature and water to ethyl acetate volume ratio (W:Ac) on
the size of salbutamol sulfate (SS) particles prepared by emulsification-diffusion (μm ± s.d.,n = 3)

| | W:Ac (ml:ml) | Average Diameter of SS particles (μm) |
|---|---|---|
| Temperature (K) | 0.8:14 | 1.28 ± 00.17 |
| 311 | 0.8:19 | 0.55 ± 0.08 |
| | 0.8:24 | 1.00 ± 0.12 |
| W:Ac ml:ml | T (K) | Average diameter of SS particles (μm) |
| 0.8:19 | 303.0 | 0.7 ± 0.10 |
| | 311.0 | 0.55 ± 0.08 |
| | 316.0 | 0.64 ± 0.07 |

(μm ± s.d., n = 3) = represent mean diameter μm and the standard deviation (±s.d.) for 3 batches

The results indicate that at constant temperature the average size of SS spheres initially decreases with an increase in the volume of ethyl acetate relative to that of water (going from 0.8:14 to 0.8:19). However, at larger volumes of ethyl acetate (on going from 0.8:19 to 0.8:24) the trend is reversed, and the size of the SS particles increases with decreasing W:Ac ratio. The same trend was observed at 303 and 316 K (results not shown). With the initial decrease in the W:Ac ratio, the amount of water per volume of (saturated) organic phase is reduced due to the solubility of water in ethyl acetate (Hefter, 1992). As emulsification conditions are kept constant, a smaller volume of the aqueous phase translates into fewer emulsion droplets, which in turn leads to fewer droplet collisions, and thus lower rates of coalescence (GalindoRodriguez, 2004). The overall effect is to reduce the size of the resulting SS particles. However, as more ethyl acetate is added and the W:Ac ratio is further reduced, there is an increase in viscosity of the dispersed aqueous phase because more water is dissolved into ethyl acetate. An increase in viscosity hinders the break-up of the emulsion droplets. This effect is opposite to, and in this case counter-balances the effect due to dilution, giving origin to SS spheres with larger diameters. Similar results have also been observed in the preparation of organic polymer particles using water-benzyl alcohol and water-propylene carbonate systems (Galindo-Rodriguez, 2004; Kwon, 2001; Leroux, 1995; Quintanar-Guerrero et al., 1996).

A similar trend was observed regarding the effect of temperature on the size of SS 20 particles at constant W:Ac. However, the effect of temperature is less pronounced than that of the volume ratio. The same rational can be used to explain both trends. As the temperature is increased, the solubility of water in ethyl acetate increases and that of ethyl acetate in water decreases (Hefter, 1992). Here again are the two competing effects of dilution and viscosity enhancement of the internal phase. It is worth noticing, however, that the reduction in interfacial tension (y) as the temperature increases does not seem to influence the trend in particle size relative to that observed for the W:Ac volume ratio.

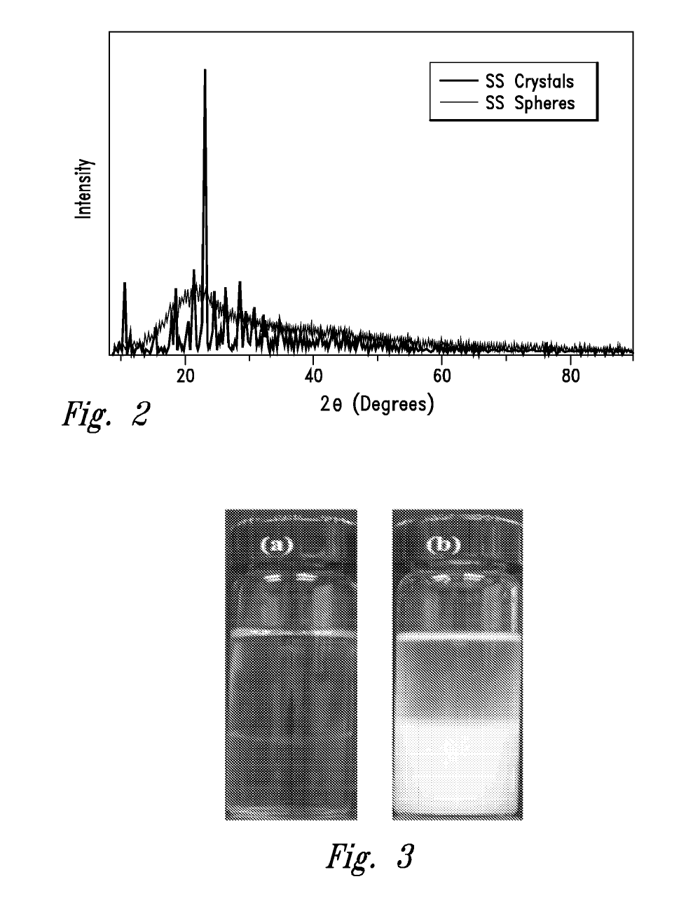

The crystallinity of commercial SS and the SS spheres prepared by emulsification-diffusion was examined by XRD. The spectra are shown in FIG. 2. The results demonstrate that, the commercial SS crystals become amorphous spheres after emulsification-diffusion. While controlled evaporation can be used to obtain large SS crystals from an aqueous solution (Begat et al., 2004), there is not enough time for the growing nuclei to crystallize during the emulsification-diffusion process. The particles are thus aggregate of multiple nuclei templated by the shape/size of the emulsion droplets.

3.1.2. Particle-Stabilized Emulsions. In the preceding section it was shown that smooth spherical particles of SS can be obtained with the emulsification-diffusion technique. It was also shown that formulation parameters including temperature and W:Ac volume ratio can be used to tune the size of the drug particles. The SEM micrographs indicate, however, that the resulting systems were very polydisperse. The broad range in particle size may be attributed to a large rate of coalescence of the emulsion droplets that is expected in the absence of a stabilizing agent at the water-ethyl acetate interface.

Surfactants can be used to control the size of particles formed by emulsification-diffusion (Choi et al., 2002; Kwon, 2001; Leroux, 1995; Quintanar-Guerrero et al., 1996; Trotta et al., 2004). In this study how the stabilization of emulsion droplets affects the size and size distribution of SS particles formed by emulsification-diffusion was investigated. However, instead of using surfactants, particle-stabilized emulsions were studied. There are several advantages in using particle-stabilized emulsions in this case. Perhaps the most important advantage is that particles (those used to stabilize the emulsion) that might be physisorbed onto the drug particle surface after emulsification-diffusion can be easily washed away. The same is not true for amphiphiles. This is very significant for pMDI-based systems because the number of excipients in FDA-approved formulations is very restricted (Courrier et al., 2002). Moreover, particles are known to impart superior stability to emulsion droplets when compared to surfactants due to the high adsorption energy at fluid-fluid interfaces (Aveyard et al., 2003; Binks, 2002; Binks and Whitby, 2005; Clegg et al., 2005; Kralchevsky et al., 2005). One disadvantage of particle-stabilized emulsions is that a generally higher energy input is necessary to form emulsions of the same droplet size as those systems containing surfactant. This happens because particles are not interfacially active in the sense of reducing the interfacial tension. Once they reach the interface, they might be strongly bound (large adsorption energy) if they are wetted by both the organic and aqueous side of the interface. However, they do not necessarily reduce the tension as surfactants do (Aveyard et al., 2003; Binks, 2002). In order to avoid such problems, surfactants can be added to help in the emulsion formation (Binks, 2002). Here, however, the organic phase was selected not only because of its relatively low toxicity (Bahl and Sah, 2000), but also because it has a low tension against water 6.8 mN·m-l (Donahue and Bartell, 1952). This allowed the formation of small emulsion droplets at low energy input, even in the absence of surfactants.

Lecithin was chosen for these studies because it is an excipient in several FDA-approved pMDI formulations (Courrier et al., 2002). The treated lecithin is insoluble in both water and ethyl acetate. However, it can form stable aqueous suspensions. The lecithin particles used here have an effective particle diameter of 270 nm and polydispersity of 0.295, as probed by DLS.

The ability of lecithin particles in stabilizing W/Ac emulsions was probed, and the results shown in FIG. 3. Both images were taken 5 min after mechanical energy (sonication) to a 40:60% W:Ac volume ratio was stopped. It can be seen that the lecithin-stabilized W/Ac emulsion (FIG. 3b) is significantly more stable to coalescence than W/Ac emulsions formed without any stabilizing agent. While in FIG. 3a two clear phases are visible, in FIG. 3b, the lower phase consists of emulsion (aqueous) droplets that have settled due to gravitational fields. Coalescence, which would have been characterized by the appearance of an excess pure aqueous phase at the bottom of the vial is not observed, indicating that the particles are indeed providing a good stability to the interface.

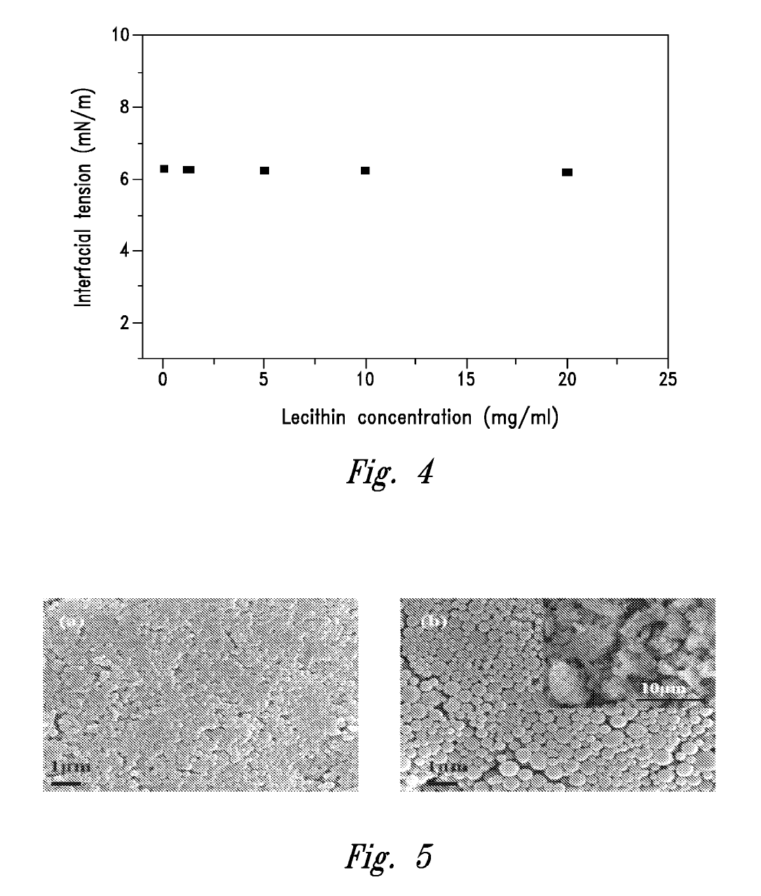

To further understand the emulsion stabilization mechanism, the interfacial tension (y) between aqueous dispersions of lecithin (saturated with ethyl acetate) against ethyl acetate (saturated with water), as a function of lecithin concentration at 298 K was also measured. The results are shown in FIG. 4.

It can be observed that the y values of the water-ethyl acetate interface in the presence of lecithin have very small deviations from the value of the bare interface (within ±1.5%). These results clearly indicate that the stabilization mechanism of the emulsion is based on the wetting of the particles at the interface; i.e., particle-stabilized emulsions, and that lecithin particles can provide good stability to the aqueous emulsion droplets in ethyl acetate.

As a consequence of this enhanced stabilization, particles of SS sulfate obtained from particle-stabilized emulsions are not only smooth and spherical (templated by the droplets), but also show significantly lower polydispersity, as shown in FIG. 5a. The size of the particles is also significantly smaller than in the absence of lecithin, with an estimated average diameter of 350 nm.

Lecithin particles that stabilize the fluid-fluid interface might be still physisorbed onto the drug surface after the particles are collected by centrifugation. The system is, therefore, washed with hexane. Stabilization studies in propellant HFAs (that will be discussed later) also indicate that lecithin particles indeed remain adsorbed at the drug surface after the preparation of the drug particles, and that the hexane wash is effective in removing the particles bound to the drug particle surface.

The methodology developed here represents a significant improvement compared to previous reports on the emulsification-diffusion technique for the formation of polar drugs (Galindo-Rodriguez, 2004). It offers an opportunity for controlling size and size distribution without the use of amphiphiles.

3.1.3. PEG-modified SS Spheres. While particle size and size distribution can be further controlled by stabilizing the W/Ac emulsion with lecithin particles, the surface morphology, and thus particle-particle interaction remains unchanged. In order to prepare stable dispersions of SS in HFAs, modification of the surface of the particles with an HFA-philic moiety was proposed. To accomplish this objective a modified version of the emulsification-diffusion method was used. The idea is to ‘trap’ an HFA-phile at the interface during the emulsification-diffusion procedure.

PEG was selected in this study for many reasons. PEG is known to have appreciable solubility in HFAs (Ridder et al., 2005; Vervaet and Byron, 1999). PEG is also widely used in the pharmaceutical industry (Otsuka et al., 2003; Schmieder et al., 2007) and an excipient in FDA-approved nasal spray formulations. Moreover, recently published ab initio calculations indicate that HFA134a interacts very favorably with the ether moiety, as that in PEG (Selvam et al., 2006; Wu et al., 2007c). Recent CPM studies also reveal that the homopolymer PEG in solution can reduce cohesive forces between drug particles in a mimicking HFA (Traini et al., 2006).

The morphology of the SS spheres modified with PEG300 from lecithin stabilized emulsion is shown in FIG. 5b. The inset FIG. 5b is a micrograph of the particles before washing. SS particles tend to strongly aggregate together before the lecithin particles are removed, while the hexane-washed SS particles were loosely packed. The average diameter of the PEG modified SS particles is estimated to be approximately 450 nm, which is smaller than those particles formed without stabilizing agents, but slightly higher than the particles obtained by the lecithin-stabilized emulsions. The polydispersity is also intermediate between the two systems. It was observed that PEG300 does not reduce the tension of the water-ethyl acetate interface. The presence of PEG in aqueous phase is expected to increase the viscosity of the internal phase, which may explain the slight increase in the size for PEG-modified SS particles compared with the case without PEG.

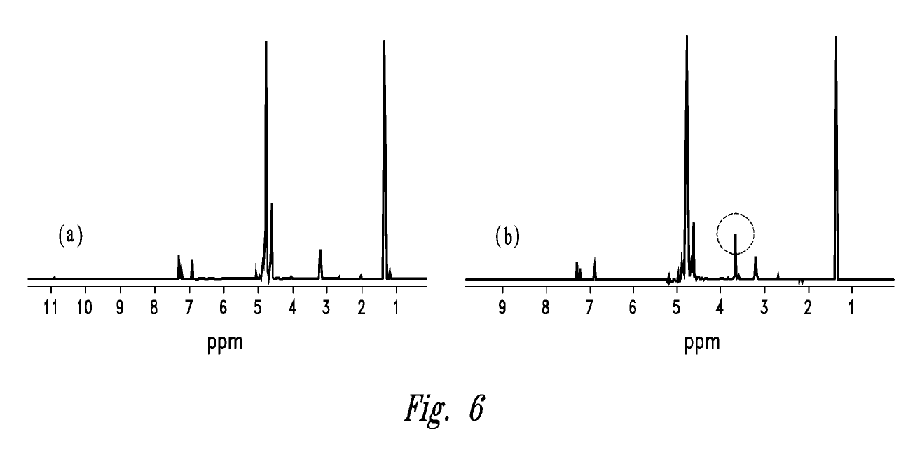

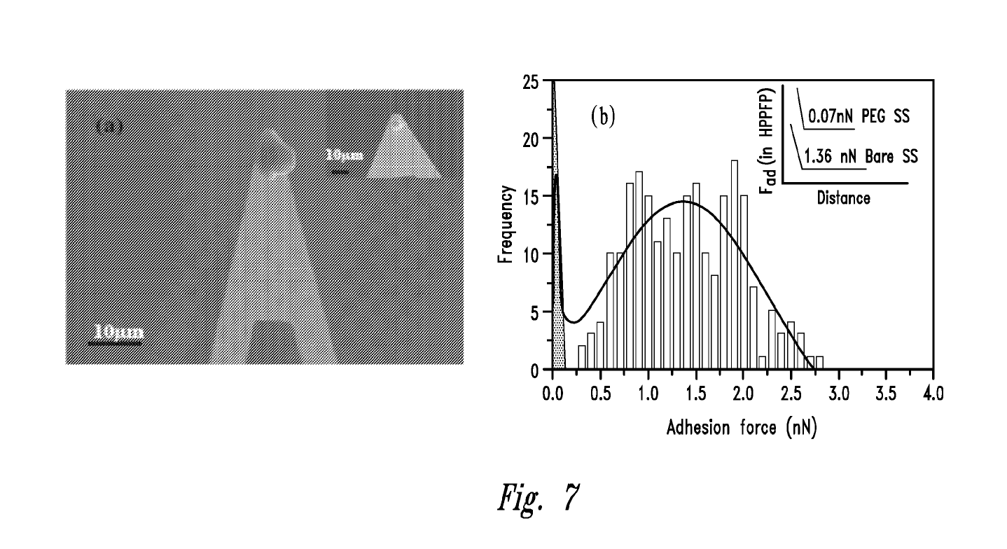

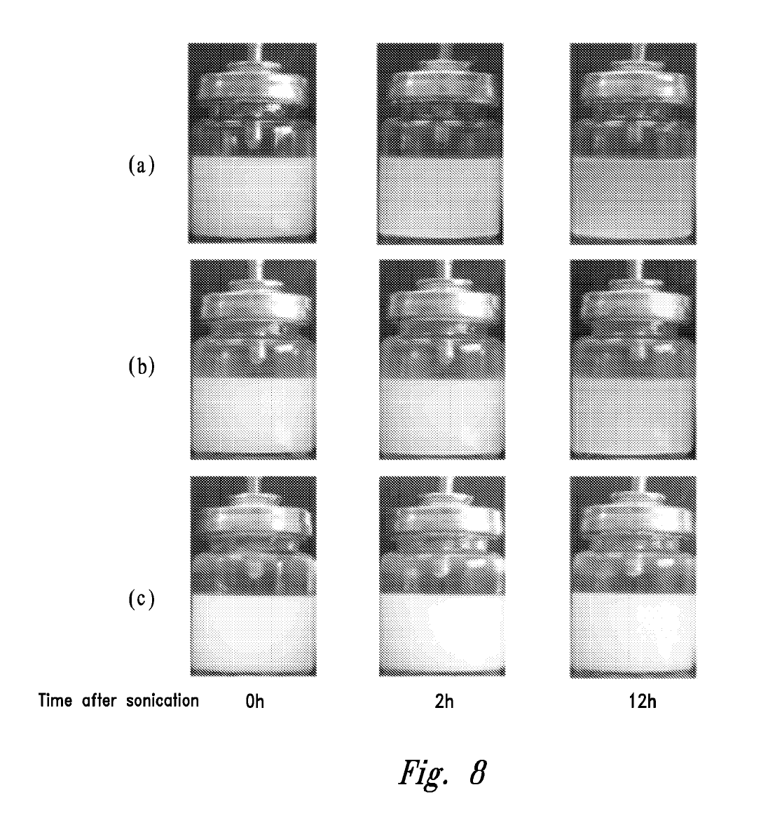

The retention of PEG on the SS particles is probed by NMR. FIGS. 6a and 6b show the IH NMR spectrum of commercial SS crystals and PEG300 modified SS spheres from lecithinstabilized W/Ac emulsions, respectively. An extra peak at 3.6 ppm (compared to pure SS) is observed. This peak is attributed to hydrogen atoms on the PEG300 chain, indicating that PEG300 molecules were trapped along with the SS spheres during the emulsification-diffusion process. From the intensity of the peaks, the molar ratio of SS to PEG300 can be calculated to be 1:0.08, which indicates that only a very small fraction of the PEG300 originally used is trapped on the particles surface, the majority being retained in the organic phase.

For the measured drug: PEG ratio, one can calculate an average of 5.2×10⁶ PEG chains per particle, which might be distributed between the surface and the bulk drug particle. Based on a 22 cross-section of a PEG chain (Gaginella, 1995), 2.9×10⁶ PEG molecules or 56% of the total would be required to fully cover a 450 nm diameter particle. The results indicate, therefore, that a large fraction of PEG (at least 40%) is actually trapped within the amorphous particle. While the NMR results unambiguously show that PEG is retained with the SS particles, the exact location (interface/core) cannot be probed by NMR alone.

3.2. Colloidal Probe Microscopy (CPM)

CPM is used to investigate the effect of PEG300 on the cohesive interactions between SS particles. SS spheres were attached to AFM cantilevers as described previously. In FIG. 7a and in the inset, SEM images of an AFM cantilever modified with a single PEG300-SS sphere are shown. Larger spheres (several microns), which are required for attachment to the AFM cantilever, were obtained simply by providing less mechanical energy during emulsification. The force of interaction (adhesion force, Fad) between the probe and particles deposited onto a silicon wafer were determined in liquid HPFP, a mimic to HFA propellants (Ashayer et al., 2004; Rogueda, 2003; Traini et al., 2006; Young et al., 2003), at 298 K. The interaction between bare SS particles, which is the baseline system ws also investigated. The CPM results for bare and PEG-modified particles are shown FIG. 7b, as Fad frequency vs. Fad. Typical (average) force curves for both systems are shown in the inset.

The average Fad between PEG-modified spheres is close to zero (0.07±0.05 nN), while that for pure SS spheres was found to be very large (1.36±1.80 nN). Based on our previous work (Wu and da Rocha, 2007), it was expected that the CPM results would correlate well with the physical stability of the formulation; i.e., for strongly cohesive system (large Fad), physical instability will ensue rapidly, while stability against flocculation is expected to be observed for systems where Fad is close to zero. It has also shown that Fad results not only correlate well with the physical stability of the formulations in HPFP, but more importantly, they could be extrapolated to the propellant HFA227. Physical stability results for bare and PEG-modified SS spheres are discussed below.

Besides providing direct information on the cohesive interaction between particles, the CPM results also answer a pending question regarding the location of PEG300. While the NMR results showed that PEG was indeed retained with the SS particles formed by the emulsification-diffusion technique, it provided no clues regarding the location—whether within the particle or at the particle surface—of the PEG groups. In view of the similar size of the particles attached to the AFM cantilevers, the fact that the bare SS spheres have a very large Fad, while the average force between PEG-modified SS particles is nearly zero indicates that a large enough fraction of the PEG retained in the particle must reside at the surface. The CPM results also show that PEG300 is strongly bound to the particle. An appreciable Fad would be otherwise observed because low molecular weight PEGs (including PEG300) show appreciate solubility in HPFP (Ridder et al., 2005; Rogueda, 2003), and would be easily removed/washed from the particle surface upon contact if not physically trapped.

PEG300 is soluble in ethyl-acetate. Time allowing, PEG300 would naturally partition to the external phase of the emulsion, thus reaching equilibrium between the aqueous droplet and the continuous ethyl acetate phases. PEG300 is also expected to be dragged towards the bulk organic phase as water diffuses out from the emulsion droplet during the emulsification-diffusion process. However, the SS particles are formed very quickly so that some of the PEG chains are expected to be ‘frozen’ within the particle core and at the particle surface, as proven by the CPM results shown above. Similar behavior has been observed for polyvinyl alcohol (PVA) at the oil/water interface, in regular (oil-in-water) emulsions. It was found out that during the diffusion process, the resulting binding of PVA to the particle surface was also very strong (nonremovable), and that was attributed to the quick hardening of particles (Galindo-Rodriguez, 2004).

While CPM results clearly indicate that there is a considerable reduction in the cohesive forces between SS spheres in HPFP when modified with PEG300, care should be exercised in extrapolating such results to propellant HFAs. HPFP is significantly larger than HFA134a and HFA227 propellant molecules, and can thus have stronger dispersive interactions with moieties of interest, including PEG300. Therefore, sedimentation rate experiments of the dispersion formulations containing SS spheres prepared by the emulsification-diffusion technique were performed, and the results discussed below.

3.3. Dispersion Stability

The stability of the SS dispersions was tested in HPFP and in the propellants HFA134a and HFA227 at saturation pressure and at 298 K. The results for HFA227 are comparable to those for HFA134a. Similar results were also obtained for HPFP. Therefore, only the results for one of the propellants (HFA134a) are discussed here. The results are summarized in FIG. 8.

As expected, bare SS spheres had poor stability in the hydrofluoroalkane solvents (FIG. 8a). Sedimentation of the particles (more dense than HFA134a) started taking place immediately after mechanical energy input stopped. A further increase in stability is observed for those particles formed with the particle-stabilized emulsions (FIG. 8b). This can be attributed to a size-reduction effect (lower gravitational forces), and the lower polydispersity of the system. Dispersions of PEG300-modified SS spheres are highly stable in the propellant HFAs (HFA227 and HFA134a), indicating that the PEG300 moiety is well solvated by the semi-fluorinated solvents. The sedimentation rate is on the order of hours and the sedimentated particles are easily resuspended simply by hand-shaking the pMDI. The bulk physical stability results follow the Fad trends determined by CPM in HPFP; i.e., the lower the Fad, the higher the physical stability of the dispersion.

It is also worth discussing the physical stability of two other propellant formulations. While PEG300-modified SS particles are very stable in HFAs, the results show that the dispersions are very weak if the particles are not washed to remove residual lecithin particles that are adsorbed at the drug particle surface. The cohesive forces between micronized SS particles in HPFP in the presence of PEG400 in solution has been recently reported (Traini et al., 2006). CPM results show that the Fad can be reduced by 26 68% in pure HPFP upon addition of various concentration of PEG400. However, our tests indicate that PEG300 in solution does not improve the stability of the formulation compared to the drug alone. Perhaps this is related to the fact that PEG is very soluble in HFA and the driving force for adsorption onto the particle surface is very weak. This is not a concern in these studies because the HFA-phile is trapped on the particle.

The applicability of the proposed methodology to another inorganic drug of interest, namely terbutaline hemisulfate (THS) was also tested. PGE300-modified THS spheres were prepared using the emulsification-diffusion technique as described above. It was found that dispersions in HFAs (HFA134a and HFA227) were of a similar (excellent) quality as those of PEG-modified SS particles discussed earlier. The positive results for THS indicate that the approach is likely general to polar drugs.

3.4. Aerosol Performance

The aerosol characteristics of the commercial VENTOLIN® HFA, bare SS, and PEGIO modified SS formulations as determined by ACI are shown in Table 2. Commercial VENTOLIN® HFA contains micronized SS crystal in HFA134a without any other excipients. This formulation is very close to the composition of the formulations proposed here. The effect of a spacer (Aerochamber Plus) is also discussed.

TABLE 2
Aerodynamic properties of Ventolin HFA ®, bare and PEG-modified SS dispersion
formulations, with or without spacer. The experiments were repeated for three
independent canisters (n = 3). The average and deviation of those results are reported
here.

| | Formulations | | | | | |
|---|---|---|---|---|---|---|
| | Ventolin HFA ® | | Bare-SS HFA134a | | PEG-SS HFA134a | |
| | (μg ± s.d., n = 3) | | (μg ± s.d., n = 3) | | (μg ± s.d., n = 3) | |
| | No | With | No | With | No | With |
| Stages | Spacer | Spacer | Spacer | Spacer | Spacer | Spacer |
| AC | 163.9 ± 10.2 | 115.7 ± 12.5 | 192.0 ± 17.6 | 164.1 ± 10.4 | 100.7 ± 8.5 | 80.3 ± 12.0 |
| SP | N/A | 332.6 ± 20.2 | N/A | 256.2 ± 20.1 | N/A | 218.4 ± 14.8 |
| IP | 459.5 ± 23.1 | 104.6 ± 13.4 | 299.1 ± 28.5 | 41.5 ± 5.4 | 249.1 ± 22.4 | 40.6 ± 5.4 |
| Stage 0 | 16.3 ± 1.0 | 12.2 ± 2.1 | 24.7 ± 1.8 | 20.0 ± 1.4 | 4.8 ± 0.3 | 4.3 ± 0.6 |
| (9.0-10.0 μm) | | | | | | |
| Stage 1 | 18.3 ± 1.6 | 16.6 ± 1.2 | 30.4 ± 3.4 | 32.2 ± 4.2 | 5.3 ± 1.0 | 5.6 ± 0.9 |
| (5.8-9.0 μm) | | | | | | |
| Stage 2 | 24.2 ± 1.5 | 22.8 ± 3.4 | 35.7 ± 2.8 | 37.3 ± 3.5 | 9.1 ± 1.1 | 6.0 ± 2.1 |
| (4.7-5.8 μm) | | | | | | |
| Stage 3 | 89.6 ± 7.6 | 95.3 ± 4.6 | 87.5 ± 6.4 | 105.3 ± 6.3 | 25.3 ± 3.6 | 48.2 ± 2.1 |
| (3.3-4.7 μm) | | | | | | |
| Stage 4 | 217.5 ± 12.4 | 281.9 ± 14.9 | 94.5 ± 9.8 | 105.9 ± 10.5 | 125.8 ± 14.5 | 111.8 ± 13.8 |
| (2.1-3.3 μm) | | | | | | |
| Stage 5 | 131.9 ± 10.1 | 163.9 ± 9.4 | 64.8 ± 8.4 | 72.3 ± 6.9 | 256.2 ± 20.4 | 245.7 ± 14.2 |
| (1.1-2.1 μm) | | | | | | |
| Stage 6 | 18.8 ± 1.2 | 21.2 ± 1.1 | 14.2 ± 0.8 | 17.0 ± 2.1 | 86.9 ± 9.8 | 78.1 ± 7.8 |
| (0.7-1.1 μm) | | | | | | |
| Stage 7 | 6.0 ± 0.6 | 8.4 ± 1.0 | 4.9 ± 2.1 | 6.5 ± 1.7 | 26.3 ± 3.5 | 32.8 ± 3.2 |
| (0.4-0.7 μm) | | | | | | |
| Filter | 3.5 ± 1.1 | 5.7 ± 2.1 | 2.6 ± 1.2 | 3.7 ± 0.8 | 9.3 ± 1.1 | 15.1 ± 1.3 |
| (0-0.4 μm) | | | | | | |
| FPF (%) | 45.9 ± 2.4 | 78.7 ± 2.9 | 39.1 ± 1.9 | 69.7 ± 2.1 | 65.3 ± 1.3 | 90.0 ± 1.6 |
| MMAD (μm) | 2.5 ± 0.1 | 2.3 ± 0.1 | 3.0 ± 0.2 | 2.8 ± 0.2 | 1.6 ± 0.2 | 1.6 ± 0.1 |
| GSD (μm) | 1.9 ± 0.1 | 1.8 ± 0.1 | 2.1 ± 0.1 | 2.1 ± 0.2 | 1.9 ± 0.1 | 1.9 ± 0.1 |

AC = actuator & valve stem;
IP = induction port;
SP = spacer;
MMAD = mass median aerosol diameter; and
GSD = geometric standard deviation;
(μg ± s.d., n = 3) = represent mean mass (μg) and the standard deviation (±s.d.) for 3 batches (n = 3).

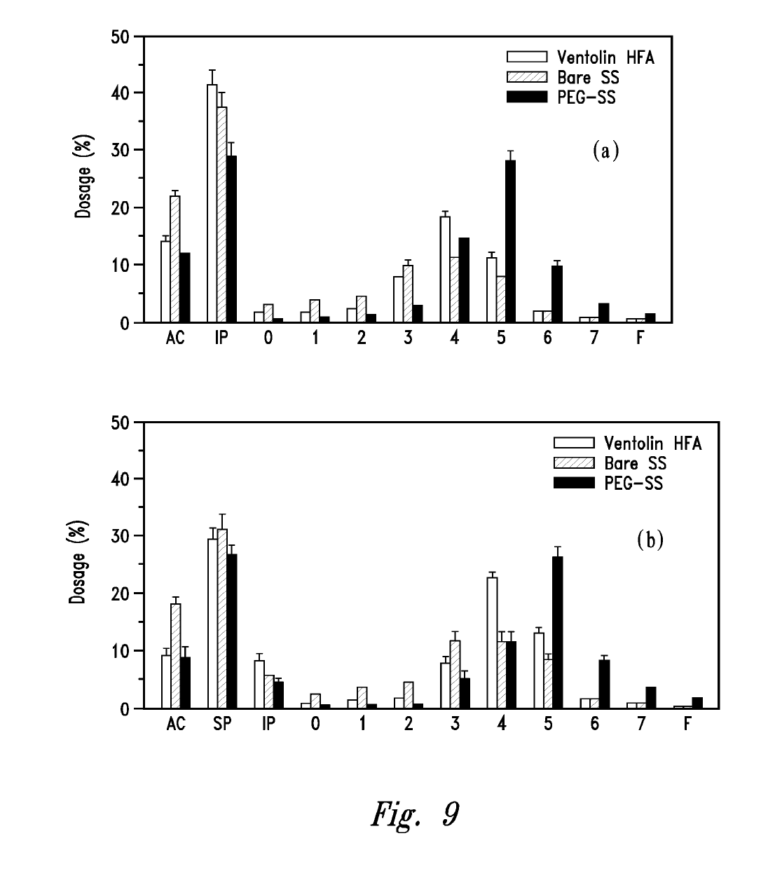

The results are summarized in FIG. 9 as the % collected in each stage relative to the total amount delivered from the pMDIs. This is done in order to facilitate the comparison among the three formulations. It can be seen from FIG. 9a that both VENTOLIN® HFA and the formulation with the bare SS spheres generate somewhat similar aerosols, where a large fraction of the drug is retained at the AC and IP (55.5 and 59.1% for VENTOLIN® HFA and bare SS formulations, respectively), in detriment to the concentration retained as FPF (stages 3-F). The use of a spacer causes a significant decrease of drug deposition in the induction port for all the formulations tested, as can be seen in FIG. 9b.

On the other hand, the PEG-modified SS formulation shows a significant improvement relative to the other two formulations. The FPF for the PEG-modified particles is approximately 20% larger than that of VENTOLIN® HFA (FPF: 65.3% vs 45.9%.). The MMAD decreased from 2.4 for the VENTOLIN® HFA to 1.5 μm for the PEG300-SS formulation. For the PEG-300 formulation, the presence of the spacer reduces the amount of drug deposited on the IP, while the FPF reaches 90.0%. It is important to note that optimization of the PEG-based formulation was not attempted (e.g.: particle size, dosage concentration, valve actuator, etc), suggesting that even better aerosols can be potentially achieved with this approach. Compared with previous work on the formation of stable SS dispersion formulations such as hollow porous particles (Dellamary et al., 2000), our formulation showed a comparable physical stability and aerosol performance, but with significantly lower concentration of excipients and high payload—nearly 100%.

In this work the applicability of a novel methodology for engineering polar drug particles with enhanced stability and aerosol characteristics in propellant HFAs was demonstrated. The approach consists in ‘trapping’ HFA-philic moieties at the surface of drug particles using a modified emulsification-diffusion method. The surface-trapped groups are shown to act as stabilizing agents, thus preventing flocculation of the otherwise unstable colloidal drug particles.

The size and polydispersity of the smooth spherical particles of a model polar drug (salbutamol sulfate, SS) generated by the emulsification-diffusion method can be controlled by varying temperature, water:oil volume ratio, and by the addition of lecithin particles, an emulsion stabilizing agent. PEG300 was selected as the candidate HFA-phile based on our previous studies that indicated that propellant HFAs can solvate well moieties containing the ether group (Selvam et al., 2006; Wu et al., 2007c). While NRM results indicated that PEG300 was indeed trapped with the polar particles formed from particle-stabilized emulsions, it provided no clue on the location of the moieties (interface vs. bulk). CPM results unambiguously demonstrate that the surface of the particles is densely populated by PEG300 chains, as indicated by a reduction in the adhesion force (Fad) from 1.36±1.80 nN for pure SS spheres, down to approximately zero (0.07±0.05 nN) for PEG-trapped SS particles. CPM studies also offer an opportunity to decouple the effect of particle-particle interactions from the other formulation variables on the performance of the aerosol.

Dispersions of the PEG-trapped SS particles in the model propellant HPFP, and in the propellant HFAs (HFA134a and HFA227) demonstrate long term physical stability. The results compared very favorably to formulations containing the SS particles without the surface modification. These results are also in excellent agreement with the CPM observations. Large Fad translate in fast creaming or sedimentation rates, while the small Fad due to the ability of PEG300-trapped moieties to screen the cohesive interactions between drug particles result in long term physical stability of the formulation. It is also noteworthy to mention that the CPM results obtained in HPFP do extrapolate to both HFA227 and HFA134a. While HPFP is generally accepted as a mimicking solvent to HFAs, it is a much larger molecule than the propellants HFA134a and HFA227. One possible difference in the behavior of these systems is that HPFP should be capable of interacting more strongly with moieties of interest (such as PEG300) through dispersion-type forces. This difference is expected to be more pronounced when compared to the smaller HFA134a than HFA227.

Formulations containing the surface-trapped HFA-philes not only showed improved physical stability, but also dramatically increased the aerosol characteristics compared to both bare SS particles made by emulsification-diffusion (the baseline system), and a commercial (micronized SS) formulation. The presence of a spacer further reduced the amount of PEG-trapped particles retained at the induction port and actuator, with a corresponding increase in FPF that reached 90%.

The proposed particle-formation methodology has several advantages compared to surfactant-stabilized colloids. No free stabilizers remain in solution, thus decreasing the risk of toxicity, and the challenges associated with the synthesis of well-balanced amphiphiles are circumvented. PEG-trapped terbutaline hemisulfate particles also showed similar bulk physical stability and aerosol performance to those described for PEG-modified SS. The results suggest this to be a generally applicable methodology to polar drugs. The approach could be also extended to the formulation of large polar molecules, and/or drug combinations.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### REFERENCES

Ashayer, R., Luckham, P. P., Manimaaran, S., Rogueda, P., 2004. Investigation of the molecular interactions in a pMDI formulation by atomic force microscopy. Colloids Surf., A 21, 533-543.

Aveyard, R., Binks, B. P., Clint, J. H., 2003. Emulsions stabilised solely by colloidal particles. Adv. Colloid Interface Sci. 100-102, 503-546.

Bahl, Y., Sah, H., 2000. Dynamic Changes in Size Distribution of Emulsion Droplets During Ethyl Acetate-Based Microencapsulation Process. AAPS Pharm Sci Tech 1, Article 5.

Begat, P., Morton, D. A. V., Staniforth, J. N., Price, R., 2004. The cohesive-adhesive balances in dry powder inhaler formulations I: Direct quantification by atomic force microscopy. Pharm. Res. 21, 1591-1597.

Binks, B. P., 2002. Particles as surfactants—similarities and differences. Curr. Opin. Colloid Interface Sci. 7, 21-41.

Binks, B. P., Whitby, c. P., 2005. Nanoparticle silica-stabilized oil-in-water emulsions: improving emulsion stability. Colloids Surf., A 253, 105-115.

Blondino, F. E., Byron, P. R., 1998. Surfactant dissolution and water solubilization in chlorinefree liquified gas propellants. Drug Dev. Ind. Pharm. 24, 935-945.

Boskabady, M. H., Saadatinejad, M., 2003. Airway Responsiveness to Beta-Adrenergic Agonist (Salbutamol) in Asthma. J. Asthma 40, 917-925.

Butt, H.-J., Cappella, B., Kappl, M., 2005. Force measurements with the atomic force microscope: Technique, interpretation and applications. Surf. Sci. Rep. 59, 1-152.

Choi, S.-W., Kwon, H.-Y., Kim, W.-S., Kim, J.-H., 2002. Thermodynamic parameters on poly(d,l-lactide-co-glycolide) particle size in emulsification-diffusion process. Colloids Surf., A 201, 283-289.

Clegg, P. S., Herzig, E. M., Schofield, A. B., Horozov, T. S., Binks, B. P., Cates, M. E., Poon, W. C. K., 2005. Colloid-stabilized emulsions: behaviour as the interfacial tension is reduced. J. Phys.: Condens. Matter 17, s3433-s3438.

Courrier, H. M., Butz, N., Vandamme, T. P., 2002. Pulmonary drug delivery system: recent developments and prospects. Crit. Rev. Therapeu. Drug Carri. Syst. 19, 425-498.

Dellamary, L. A., Tarara, T. E., Smith, D. J., Woelk, C. H., Adractas, A., Costello, M. L., Gill, H., Weers, J. G., 2000. Hollow porous particles in metered dose inhalers. Pharm Res 17, 168174.

Dickinson, P. A. H., Wyn, S., Kellaway, L. W., 2001. Novel nanoparticles for pulmonary drug administration. J. Drug Targeting 9, 295-302.

Donahue, D. J., Bartell, F. E., 1952. The boundary tension at water-organic liquid interfaces. J. Phys. Chern. 56, 480-484.

Edwards, D. A., Hanes, J., Caponetti, G., Hrkach, J., Ben-Jebria, A., Eskew, M. L., Mintzes, J., Deaver, D., Lotan, N., Langer, R., 1997. Large porous particles for pulmonary drug delivery. Science 276, 1868-1871.

Gaginella, T. S., 1995. Handbook of Methods in Gastrointestinal Pharmacology. CRC Press, Boca Raton.

Galindo-Rodriguez, S. A., Eric; Fessi, Hatem; Doelker, Eric., 2004. Physicochemical Parameters Associated with Nanoparticle Formation in the Salting-Out, Emulsification-Diffusion, and Nanoprecipitation Methods. Pharm. Res. 21, 1428-1439.

Hefter, G T., 1992. Solubility data. Ethyl acetate-water system. Solubility Data Series 48, 60126.

James, S. S., 2002. Novel excipients for inhalation drug deliver: expanding the capability of the MDL Drug Delivery Tech. 2, 62, 64-69.

Jones, S. A., Martin, G. P., Brown, M. B., 2006. Stabilization of deoxyribonuclease in hydrofluoroalkanes using miscible vinyl polymers. J. Controlled Release 115, 1-8.

Kralchevsky, P. A., Ivanov, L B., Ananthapadmanabhan, K. P., Lips, A., 2005. On the Thermodynamics of Particle-Stabilized Emulsions: Curvature Effects and Catastrophic Phase Inversion. Langmuir 21, 50-63.

Kwon, H.-Y. L., J.-Y.; Choi, S.-W.; Jang, Y.; Kim, J.-H., 2001. Preparation of PLGA nanoparticles containing estrogen by emulsification-diffusion method. Colloids Surf., A 182, 123-130.

Lafrate, R. P., Massey, K. L., Hendeles, L., 1986. Current concepts in clinical therapeutics: asthma. Clin. Pharm. 5, 206-227. Leroux, J. C. A., Eric; Doelker, Eric; Gurny, Robert., 1995. New approach for the preparation of nanoparticles by an emulsification-diffusion method. Em. J. Pharm. Biopharm. 41, 14-18.

Liao, Y.-H., Brown, M. B., Jones, S. A., Nazir, T., Martin, G. P., 2005. The effects of polyvinyl alcohol on the in vitro stability and delivery of spray-dried protein particles from surfactant free HFA 134a-based pressurised metered dose inhalers. Int. J. Pharm. 304, 2939.

McDonald, K. J., Martin, G. P., 2000. Transition to CFC-free metered dose inhalers -into the new millennium. Int. J. Pharm. 201, 89-107.

Noakes, T. J., 1995. CFC's their replacement and the ozone layer. J. Aerosol Med. 8, 3-7. Otsuka,

H., Nagasaki, Y., Kataoka, K., 2003. PEGylated nanoparticles for biological and pharmaceutical applications. Adv. Drug Delivery Rev. 55, 403-419.

Quintanar-Guerrero, D., Fessi, H., Allemann, E., Doelker, E., 1996. Influence of stabilizing agents and preparative variables on the formation of poly(D,L-lactic acid) nanoparticles by an emulsification-diffusion technique. Int. J. Pharm. 143, 133-141.

Ranucci, J. A., Dixit, S., Bray, R N., Jr., Goldman, D., 1990. Controlled flocculation in metereddose aerosol suspensions. Pharm. Res 14, 68-74.

Ridder, K. B., Davies-Cutting, C. J., Kellaway, L. W., 2005. Surfactant solubility and aggregate orientation in hydrofluoroalkanes. Int. J. Pharm. 295, 57-65.

Rogueda, P., 2005. Novel hydrofluoroalkane suspension formulations for respiratory drug delivery. Expert Opin. Drug Delivery 2, 625-638.

Rogueda, P. G. A., 2003. HPFP, a Model Propellant for pMDIs. Drug Dev. Ind. Pharm. 29, 39, 49.

Schmieder, A. H., Grabski, L. E., Moore, N. M., Dempsey, L. A., Sakiyama-Elbert, S. E., 2007. Development of novel poly(ethylene glycol)-based vehicles for gene delivery. Biotechnol. Bioeng. 96, 967-976.

Selvam, P., Peguin, R. P. S., Chokshi, D., da Rocha, S. R. P., 2006. Surfactant Design for the 1,1,1,2-Tetrafluoroethane-Water Interface: ab initio Calculations and in situ HighPressure Tensiometry. Langmuir 22, 8675-8683.

Smyth, H. D. C., Beck, V. P., Williams, D., Hickey, A. J., 2004. The influence of formulation and spacer device on the in vitro performance of solution chlorofluorocarbon-free propellant driven metered dose inhalers. AAPS Pharm Sci Tech 5, 1-7.

Steckel, H., Wehle, S., 2004. A novel formulation technique for metered dose inhaler (MDI) suspensions. Int. J. Pharm. 284, 75-82. Telko, M. J., Hickey, A. J., 2005. Dry powder inhaler formulation. Respir. care 50, 1209-1227.

Traini, D., Young, P. M., Rogueda, P., Price, R., 2006. Investigation into the influence of polymeric stabilizing excipients on inter-particulate forces in pressurised metered dose inhalers. Int. J. Pharm. 320, 58-63.

Trotta, M., Chirio, D., Cavalli, R, Peira, E., 2004. Hydrophilic Microspheres from Water-in-Oil Emulsions by the Water Diffusion Technique. Pharm. Res. 21, 1445-1449.

Tzou, T.-Z., Pachuta, R. R, Coy, R B., Schultz, R K., 1997. Drug from selection in Albuterol Containing Metered-Dose inhaler formulation and its impact on chemical and physical stability. J. Pharm. Sci. 86, 1352-1357.

Vervaet, C., Byron, P. R., 1999. Drug-surfactant-propellant interactions in HFA-formulations.

Int. J. Pharm. 186, 13-30. Williams, R O J., Liu, J., 1999. Formulation of a protein with propellant HFA 134a for aerosol delivery. Eur. J. Pharm. Sci. 7, 137-144.

Williams, R O J., Patel, A. M., Barron, M. K., Rogers, T. L., 2001. Investigation of some commercially available spacer devices for the delivery of glucocorticoid steroids from a pMDI. Drug Dev. Ind. Pharm. 27, 401-412.

Wu, L., Bharatwaj, B., Panyam, J., da Rocha, S., 2007a. Novel Propellant-driven Core-Shell Formulations for the Delivery of Polar Drugs through Pulmonary Route. Pharm. Res. To IObepublished.

Wu, L., da Rocha, S., 2007. Biocompatible and Biodegradable Copolymer Stabilizers for Hydrofluoroalkane Dispersions: A Colloidal Probe Microscopy Investigation. Langmuir ASAP.

Wu, L., Peguin, R. P. S., da Rocha, S. R. P., 2007b. Understanding Solvation in 15 Hydrofluoroalkanes: Ab initio Calculations and Chemical Force Microscopy. J. Phys. Chern. Bill 8096-8104.

Wu, L., Peguin, R. P. S., Selvam, P., Chokshi, U., da Rocha, S. R P., 2007c. Molecular Scale Behavior in Alternative Propellant-Based Inhaler Formulations. Inhalation Aerosols: Physical and Biological Basis for Therapy; by Hickey, A. J., 3rd Edition, 373-397.

Young, P. M., Price, R., Lewis, D., Edge, S., Traini, D., 2003. Under pressure: predicting pressurized metered dose inhaler interactions using the atomic force microscope. J. Colloid Interface Sci. 261, 298-302.

1. A method of producing a stable dispersion of a polar drug in hydrofluoroalkane (HFA), comprising:
providing a polar drug particle; and
adding a quantity of said HFA to said polar drug particle to produce said stable dispersion.

2. The method of claim 1, further comprising sonicating said quantity of said HFA and said polar drug particle.

3. The method of claim 1, wherein said polar drug particle is produced by emulsification-diffusion.

4. The method of claim 1, wherein said polar drug particle is selected from the group consisting of a polar drug particle without a stabilizing agent, a particle-stabilized polar drug particle, an HFA-philic moiety-modified, particle-stabilized polar drug particle and combinations thereof.

5. The method of claim 4, wherein said polar drug particle without said stabilizing agent is produced by:
dissolving said polar drug in water to form an aqueous solution;
adding said aqueous solution to a first quantity of ethyl acetate;
emulsifying said aqueous solution and said first quantity of ethyl acetate to form a water-in-ethyl acetate (W/Ac) emulsion; and transferring said W/Ac emulsion to a second quantity of ethyl acetate, whereby said polar drug particle without said stabilizing agent is formed.

6. The method of claim 4, wherein said particle-stabilized polar drug particle is produced by:
providing an aqueous dispersion of a stabilizing particle;
dissolving said polar drug in said aqueous dispersion of said stabilizing particle to form a polar drug and stabilizing particle dispersion;
adding said polar drug and stabilizing particle dispersion to a first quantity of ethyl acetate;
emulsifying said polar drug and stabilizing particle dispersion and said first quantity of ethyl acetate to form a water-in-ethyl acetate (W/Ac) emulsion; and
transferring said W/Ac emulsion to a second quantity of ethyl acetate, whereby said particle-stabilized polar drug particle is formed.

7. The method of claim 6, wherein the particle is lecithin.

8. The method of claim 4, wherein said HFA-philic moiety-modified, particle-stabilized polar drug particle is produced by:
providing an aqueous dispersion of a stabilizing particle;
dissolving a quantity of said HFA-philic moiety and said polar drug in said aqueous dispersion of said stabilizing particle to form a HFA-philic moiety, polar drug and stabilizing particle dispersion;
adding said HFA-philic moiety, polar drug and stabilizing particle dispersion to a first quantity of ethyl acetate;
emulsifying said HFA-philic moiety, polar drug and stabilizing particle dispersion and said first quantity of ethyl acetate to form a water-in-ethyl acetate (W/Ac) emulsion; and
transferring said W/Ac emulsion to a second quantity of ethyl acetate, whereby said HFA-philic moiety-modified, particle-stabilized polar drug particle is formed.

9. The method of claim 8, wherein said HFA-philic moiety is a polyethylene (PEG).

10. The method of claim 1, wherein said polar drug is a pulmonary drug.

11. The method of claim 1, wherein said pulmonary drug is salbutamol sulfate or terbutaline hemisulfate.

12. The method of claim 11, wherein said pulmonary drug is a drug for the treatment of asthma.

13. The method of claim 1, wherein the HFA is selected from the group consisting of 1,1,1,2-tetrafluoroethane, 1,1,1,2,3,3,3-heptafluoropropane, and combinations thereof.

14. A composition comprising a stable dispersion of a polar drug in hydrofluoroalkane (HFA).

15. (canceled)

16. The composition of claim 14, wherein said polar drug is a pulmonary drug.

17. The composition of claim 16, wherein said pulmonary drug is salbutamol sulfate or terbutaline hemisulfate.

18. The composition of claim 16, wherein said pulmonary drug is a drug for the treatment of asthma.

19. The composition of claim 14, wherein the HFA is selected from the group consisting of 1,1,1,2-tetrafluoroethane, 1,1,1,2,3,3,3-heptafluoropropane, and combinations thereof.

20. (canceled)

21. (canceled)

22. (canceled)

23. A polar drug particle, wherein the polar drug particle is (i) without a stabilizing agent, (ii) is particle-stabilized, or (iii) is a hydrofluoroalkane(HFA)-philic moiety-modified, particle-stabilized polar drug particle,
wherein if without a stabilizing agent the polar drug particle is produced by:
dissolving said polar drug in water to form an aqueous solution;
adding said aqueous solution to a first quantity of ethyl acetate;
emulsifying said aqueous solution and said first quantity of ethyl acetate to form a water-in-ethyl acetate (W/Ac) emulsion; and
transferring said W/Ac emulsion to a second quantity of ethyl acetate, whereby said polar drug particle without said stabilizing agent is formed;
wherein if particle-stabilized, the polar drug particle is produced by:
providing an aqueous dispersion of a stabilizing particle;
dissolving said polar drug in said aqueous dispersion of said stabilizing particle to form a polar drug and stabilizing particle dispersion;
adding said polar drug and stabilizing particle dispersion to a first quantity of ethyl acetate;
emulsifying said polar drug and stabilizing particle dispersion and said first quantity of ethyl acetate to form a water-in-ethyl acetate (W/Ac) emulsion; and
transferring said W/Ac emulsion to a second quantity of ethyl acetate, whereby said particle-stabilized polar drug particle is formed; and
wherein if hydrofluoroalkane(HFA)-philic moiety-modified, particle-stabilized, the polar drug particle is produced by:
providing an aqueous dispersion of a stabilizing particle;
dissolving a quantity of said HFA-philic moiety and said polar drug in said aqueous dispersion of said stabilizing particle to form a HFA-philic moiety, polar drug and stabilizing particle dispersion;
adding said HFA-philic moiety, polar drug and stabilizing particle dispersion to a first quantity of ethyl acetate;
emulsifying said HFA-philic moiety, polar drug and stabilizing particle dispersion and said first quantity of ethyl acetate to form a water-in-ethyl acetate (W/Ac) emulsion; and
transferring said W/Ac emulsion to a second quantity of ethyl acetate, whereby said HFA-philic moiety-modified, particle-stabilized polar drug particle is formed.
